(19) United States
(12) Patent Application Publication
(10) Pub. No.: US 2025/0302677 A1
(43) Pub. Date: Oct. 2, 2025

(54) Diaper With Cleaning Wipe Dispenser

(71) Applicant: Kaneesha Chanteria Henderson, Athens, GA (US)

(72) Inventor: Kaneesha Chanteria Henderson, Athens, GA (US)

(21) Appl. No.: 19/092,550

(22) Filed: Mar. 27, 2025

Related U.S. Application Data
(60) Provisional application No. 63/570,555, filed on Mar. 27, 2024.

(51) Int. Cl. A61F 13/84; A61F 13/496; A61F 13/505; A61F 13/551; A61F 13/56

(52) U.S. Cl. CPC A61F 13/84; A61F 13/496; A61F 13/505; A61F 13/55105; A61F 13/5638; A61F 2013/8476

(57) ABSTRACT

A diaper includes a body with a front portion, a back portion, and a middle portion disposed therebetween. A wipe assembly includes at least one wipe and a container in which the at least one wipe is disposed. The wipe assembly is secured to the body portion of the diaper.

### CROSS-REFERENCE TO RELATED APPLICATIONS

This application claims priority to and the benefit of the filing date of U.S. Provisional Patent Application No. 63/570,555, filed Mar. 27, 2024, the entirety of which is hereby incorporated by reference herein.

### FIELD

The application is generally related to diapers and, more particularly, to diapers including a dispenser for one or more cleaning wipes to facilitate ease of changing a diaper.

### BACKGROUND

Typically, when changing a diaper on a baby, some form of cleanup is desired, either of the baby, the person changing the baby's diaper, or both. Of course, it is known to carry diapers, wipes, ointments, bottles, etc., in a baby bag, backpack, purse, etc., but it may not always be feasible, or desirable, to carry the entire ensemble of baby care products. For example, various facilities such as, but not limited to, airports, sporting event venues, etc., may limit the number and/or size of bags allowed to enter. In such instances, there is the need to limit the number of different items that an individual may have to have on their person to effect the successful changing of a baby's soiled diaper.

From the foregoing, one recognizes the need for sanitary wipes and methods of use thereof that facilitate the ease and level of sanitation of changing a diaper on a user.

### SUMMARY

Embodiments of the present disclosure preferably include a diaper having a body portion including a front portion, a back portion, and a middle portion disposed therebetween. To accommodate efficiently providing cleaning wipes, a wipe assembly including a flexible container in which at least one wipe is disposed is secured to the body portion of the diaper, optionally on the outer surface of the body portion. In this manner, wipes for cleaning when changing a baby's diaper need not be carried in an additional bag, purse etc., as they are secured to the diaper. Such assemblies may also be used for the provision of ointments, gels, etc.

Additional advantages of the invention will be set forth in part in the description that follows, and in part will be obvious from the description, or may be learned by practice of the invention. The advantages of the invention will be realized and attained by means of the elements and combinations particularly pointed out in the appended claims. It is to be understood that both the foregoing general description and the following detailed description are exemplary and explanatory only and are not restrictive of the invention, as claimed.

### DESCRIPTION OF THE DRAWINGS

These and other features of the preferred embodiments of the invention will become more apparent in the detailed description in which reference is made to the appended drawings wherein:

FIG. 1 is a top plan view of a diaper with a cleaning wipe dispenser in accordance with an embodiment of the present disclosure; and

FIG. 2A and 2B are rear plan views of an alternate embodiment of a diaper with a cleaning wipe dispenser in accordance with the present disclosure, with the wipe dispenser in the sealed and the unsealed condition, respectively.

### DETAILED DESCRIPTION

The present invention now will be described more fully hereinafter with reference to the accompanying drawings, in which some, but not all embodiments of the invention are shown. Indeed, this invention may be embodied in many different forms and should not be construed as limited to the embodiments set forth herein; rather, these embodiments are provided so that this disclosure will satisfy applicable legal requirements. Like numbers refer to like elements throughout. It is to be understood that this invention is not limited to the particular methodology and protocols described, as such may vary. It is also to be understood that the terminology used herein is for the purpose of describing particular embodiments only, and is not intended to limit the scope of the present invention.

Many modifications and other embodiments of the invention set forth herein will come to mind to one skilled in the art to which the invention pertains having the benefit of the teachings presented in the foregoing description and the associated drawings. Therefore, it is to be understood that the invention is not to be limited to the specific embodiments disclosed and that modifications and other embodiments are intended to be included within the scope of the appended claims. Although specific terms are employed herein, they are used in a generic and descriptive sense only and not for purposes of limitation.

As used throughout, the singular forms “a,” “an,” and “the” include plural referents unless the context clearly dictates otherwise. Thus, for example, unless the context dictates otherwise, reference to “a tab” provides disclosure of embodiments in which only a single such tab is provided, as well as embodiments in which a plurality of such tabs are provided.

All technical and scientific terms used herein have the same meaning as commonly understood to one of ordinary skill in the art to which this invention belongs unless clearly indicated otherwise.

As used herein, the terms “optional” or “optionally” mean that the subsequently described event or circumstance may or may not occur, and that the description includes instances where said event or circumstance occurs and instances where it does not.

As used herein, the term “at least one of” is intended to be synonymous with “one or more of.” For example, “at least one of A, B and C” explicitly includes only A, only B, only C, and combinations of each.

Ranges can be expressed herein as from “about” one particular value, and/or to “about” another particular value. When such a range is expressed, another aspect includes from the one particular value and/or to the other particular value. Similarly, when values are expressed as approximations, by use of the antecedent “about,” it will be understood that the particular value forms another aspect. It will be further understood that the endpoints of each of the ranges are significant both in relation to the other endpoint, and independently of the other endpoint. Optionally, in some aspects, when values are approximated by use of the antecedents “about,” “substantially,” or “generally,” it is contemplated that values within up to 15%, up to 10%, up to 5%, or up to 1% (above or below) of the particularly stated value can be included within the scope of those aspects. In other aspects, when angular values are approximated by use of the antecedents “about,” “substantially,” or “generally,” it is contemplated that angular values within up to 15degrees, up to 10 degrees, up to 5 degrees, or up to one degree (above or below) of the particularly stated angular value can be included within the scope of those aspects.

In the following description and claims, wherever the word “comprise” or “include” is used, it is understood that the words “comprise” and “include” can optionally be replaced with the words “consists essentially of” or “consists of” to form another embodiment.

It is to be understood that unless otherwise expressly stated, it is in no way intended that any method set forth herein be construed as requiring that its steps be performed in a specific order. Accordingly, where a method claim does not actually recite an order to be followed by its steps or it is not otherwise specifically stated in the claims or descriptions that the steps are to be limited to a specific order, it is in no way intended that an order be inferred, in any respect. This holds for any possible non-express basis for interpretation, including: matters of logic with respect to arrangement of steps or operational flow; plain meaning derived from grammatical organization or punctuation; and the number or type of aspects described in the specification.

The following description supplies specific details in order to provide a thorough understanding. Nevertheless, the skilled artisan would understand that the apparatus, system, and associated methods of using the apparatus can be implemented and used without employing these specific details. Indeed, the apparatus, system, and associated methods can be placed into practice by modifying the illustrated apparatus, system, and associated methods and can be used in conjunction with any other apparatus and techniques conventionally used in the industry.

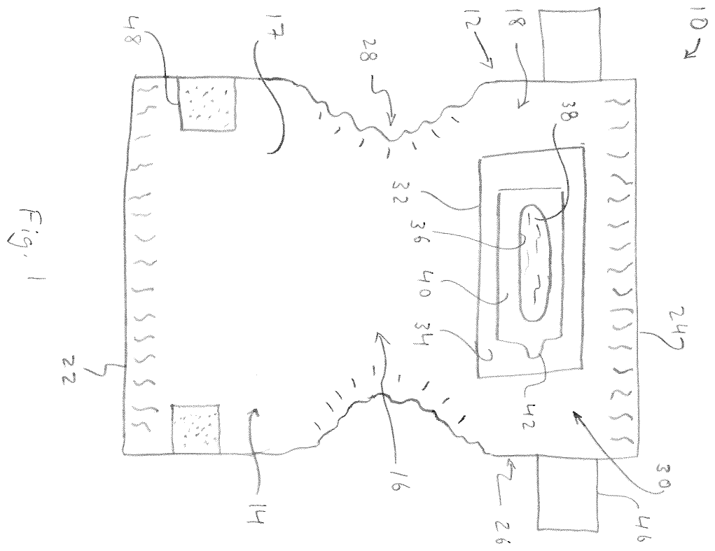

Referring now to the Figures, a diaper 10 including a cleaning wipe dispenser assembly 30 in accordance with an embodiment of the present disclosure is shown. Referring specifically to FIG. 1, the diaper 10 includes a body portion 12 having a front portion 14 that is configured to be disposed on the front side of a baby, a back portion 18 that is configured to be disposed on the back side of a baby 18, and the middle portion 16 that is disposed therebetween. The diaper 10 includes a front top edge 22 and a back top edge 24, the ends of which are connected by a pair of side edges 26 extending therebetween. As shown, each side edge 26 includes a leg cut-out 28 so that when laid flat, the diaper 10 has an hour glass like shape. In some aspects, the top front edge 22, back top edge 24, and leg cut-outs 28 can each be elasticized to ensure a snug fit when the diaper 10 is fitted on a baby. As shown, a fastener assembly including a pair of hook-and-loop fasteners having tabs 46 and corresponding patches 48 are disposed on the side edges 26 of the front and back portions 12 and 18 of the diaper 10, respectively. The hook-and-loop tabs 46 and patches 48 may be used to adjustably secure the diaper 10 on the baby, and are reusable so that the diaper 10 may be adjusted multiple times. In alternate embodiments, the hook-and-loop tabs 46 and patches 48 may be replaced by adhesive tabs, as is known in the art. As shown, the diaper 10 also includes a wipe assembly 30 disposed on an outer surface 17 of the body portion 12.

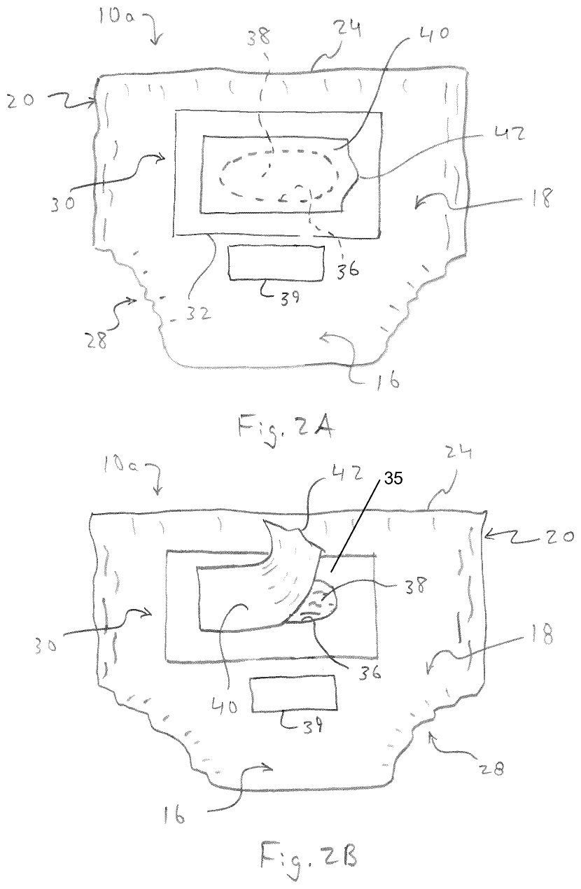

Referring additionally to FIGS. 2A and 2B, the wipe assembly 30 is preferably disposed on the outer surface 17 of the back portion 18 of the diaper's body portion 12, thereby making it harder for the wipe assembly 30 to be grasped by the baby on which the diaper is fitted. In the embodiment shown, the wipe assembly 30 includes a wipe container 32 formed by a top panel 34 that defines in aperture 36 (optionally, an elongated aperture) therein. Preferably, the container 32 is formed by a waterproof flexible packet, such as a foil or plastic packet, in which one or a plurality of wipes 38 may be stored. Accordingly, in some aspects, the wipe container 32 can cooperatively be formed by the outer surface 17 of the body portion 12 and the top panel 34. In other aspects, a bottom panel can be positioned between the top panel and the outer surface 17 of the body portion 12 so that the top panel 34 and the bottom panel cooperate to form the waterproof flexible packet. In further additional aspects, the wipe container 32 can be embedded within the body portion 12 of the diaper 10 so that at least a portion of the wipe container is disposed behind the outer surface 17.

A cover panel 40 can removably extend cross the aperture 36. The cover panel can, for example, comprise a flexible panel that is adhered to the outer surface 35 of the top panel 34 of the container 32 so that the aperture 36 is sealed in a waterproof manner. Although the seal need not be waterproof, it can be advantageous in that most cleaning wipes 38 that are used on babies are typically moistened. Accordingly, optionally, in these aspects, the one or plurality of wipes 38 can be moistened wipes. In further aspects, the moistened wipes can be impregnated with one or more of a disinfectant or a skin hydration substance. As best seen in FIG. 2B, the cover panel 40 can include a pull tab 42 to assist in partially pulling the flexible panel 40 away from the outer surface of the top panel 34 so that individual wipes 38 may be removed from the container 32 through the aperture 36. The flexible panel 40 can be resealable to the outer surface of the top panel 34 of the container 32 to prevent the other wipes 38 from drying out. The flexible panel 40 may be either opaque or transparent.

Note, as shown in FIGS. 2A and 2B, in alternate embodiments, the wipe assembly 30 may be disposed on diapers 10a that are of the pull-up variety, having a continuous waste band 20. Additionally, embodiments of the wipe assembly 30 may be utilized on protective undergarments for use on individuals in adulthood rather than just babies and small children. As well, in yet other alternate embodiments, additional containers, such as a flexible ointment packet 39 may be disposed on the outer surface of the corresponding 10a diaper to facilitate those instances in which additional treatment products are required beyond the cleaning wipes 38. The ointment pack can comprise, for example, a notch to promote tearing the ointment pack or a removable cap or seal to access the ointment therein. Optionally, the ointment pack can be resealable. For example, the seal can comprise a replaceable flap. In additional aspects, the cap can be reattached (e.g., via one or more threads).

Also disclosed herein is a kit comprising a plurality of diapers 10. The kit can further comprise a packaging within which the plurality of diapers 10 are received. For example, the packaging can comprise polymer such as shrink wrap or a box such as a paper or cardboard box.

Although the foregoing invention has been described in some detail by way of illustration and example for purposes of clarity of understanding. certain changes and modifications may be practiced within the scope of the appended claims.

What is claimed is:

1. A diaper comprising:
a body portion including a front portion, a back portion, and a middle portion disposed therebetween; and
a wipe assembly comprising:
at least one wipe; and
a container in which the at least one wipe is disposed,
wherein the wipe assembly is secured to the body portion of the diaper.

2. The diaper of claim 1, wherein the container of the wipe assembly comprises a flexible packet secured to an outer surface of the back portion of the body portion of the diaper.

3. The diaper of claim 2, wherein the flexible packet includes an aperture defined in a top panel of the packet, and a flexible panel that selectively covers the aperture.

4. The diaper of claim 3, wherein the flexible panel is resealable with a portion of an outer surface of the top panel of the flexible packet.

5. The diaper of claim 3, further comprising a fastener assembly that is configured to secure a pair of side edges of the back portion adjacent a pair of side edges of the front portion.

6. The diaper of claim 5, wherein the fastener assembly further comprises one of a pair of tape fasteners and a pair of hook-and-loop fasteners.

7. The diaper of claim 1, wherein the diaper is a pull-up style diaper with a continuous waistband.

8. The diaper of claim 1, wherein the at least one wipe comprises at least a first damp wipe add a second damp wipe.

9. The diaper of claim 1, further comprising an ointment pocket secured to the outer surface of the body portion of the diaper.

10. The diaper of claim 1, wherein the container comprises a top panel that cooperates with the outer surface of the body portion to enclose the wipe.

11. The diaper of claim 1, wherein the container is at least partially embedded within the body portion.

12. A kit comprising a plurality of diapers as in claim 1.

13. The kit of claim 12, further comprising a packaging within which the plurality of diapers are disposed.
